(19) United States
(12) Patent Application Publication
(10) Pub. No.: US 2025/0143812 A1
(43) Pub. Date: May 8, 2025

(54) ROBOTIC CATHETER SYSTEM AND METHOD OF REPLAYING TARGETING TRAJECTORY

(71) Applicants: CANON U.S.A., INC., Melville, NY (US); THE BRIGHAM AND WOMEN'S HOSPITAL INCORPORATED, Boston, MA (US)

(72) Inventors: Fumitaro Masaki, Brookline, MA (US); Brian Ninni, Woburn, MA (US); Franklin King, Boston, MA (US); Nobuhiko Hata, Waban, MA (US)

(21) Appl. No.: 18/838,332

(22) PCT Filed: Feb. 13, 2023
(86) PCT No.: PCT/US2023/062508

§ 371 Date: Aug. 14, 2024

Related U.S. Application Data
(60) Provisional application No. 63/309,977, filed on Feb. 14, 2022.

(51) Int. Cl. A61B 34/30; A61B 1/267; A61B 34/00

(52) U.S. Cl. CPC A61B 34/30; A61B 1/2676; A61B 34/25; A61B 2034/301

(57) ABSTRACT

A system for, display controller connected to, and a method of, operating a robotic catheter system which is configured to manipulate a catheter having one or more bending segments along the catheter's length and a catheter tip at the distal end thereof, and which includes an actuator unit coupled to the bending segments via one or more drive wires arranged along a wall of the catheter. The method comprising: inserting at least part of the catheter into a lumen along an insertion trajectory that spans from an insertion point to a target; causing the actuator unit to actuate at least one of the one or more drive wires to align the catheter tip with the target; determining the position and/or orientation of the catheter tip with respect to the target; and displaying information about an accuracy of alignment between the catheter tip with respect to the target.

### CROSS-REFERENCE TO RELATED APPLICATIONS

The present application claims priority to U.S. provisional application No. 63/309,977, filed Feb. 14, 2022. The disclosure of the above-listed provisional application is hereby incorporated by reference in its entirety for all purposes. Priority benefit is claimed under 35 U.S.C. § 119(e).

### BACKGROUND INFORMATION

### Field of Disclosure

The present disclosure relates to medical devices. More particularly, the disclosure is directed to robotic catheter systems and control methods for accurately aligning the catheter tip of a steerable catheter to an intended target.

### Description of Related Art

Robotic catheters or endoscopes include a flexible tubular shaft operated by an actuating force (pulling or pushing force) applied through drive wires arranged along the tubular shaft and controlled by an actuator unit. The flexible tubular shaft (herein referred to as a “steerable catheter”) may include multiple articulated segments configured to continuously bend and turn in a snake-like fashion. Typically, the steerable catheter is inserted through a natural orifice or small incision of a patient's body, and is advanced through a patient's bodily lumen (not shown) to reach a target site, for example, a site within the patient's anatomy designated for an intraluminal procedure, such as an ablation or a biopsy. A handheld controller (e.g., a joystick or gamepad controller) may be used as an interface for interaction between the user and the robotic system to control catheter navigation within the patient's body.

The navigation of a steerable catheter can be guided by the live-view of a camera or videoscope arranged at the distal tip of the catheter shaft. To that end, a display device, such as a liquid crystal display (LDC) monitor provided in a system console or attached to a wall, displays an image of the camera's field of view (FOV image) to assist the user in navigating the steerable catheter through the patient's anatomy to reach the target site. The orientation of the camera view, the coordinates of the handheld controller, and the pose or shape of the catheter are mapped (calibrated) before inserting the catheter into the patient's body. As the user manipulates the catheter inside the patient's anatomy, the camera transfers the camera's FOV image to the display device. Ideally, the displayed image should allow the user to relate to the endoscopic image as if the user's own eyes were actually inside the endoscope cavity.

Robotic bronchoscopes as described above are increasingly used to screen patients for peripheral pulmonary lesions (PPL) related to lung cancer. See, for example, non-patent literature document 1 (NPL1), by Fielding, D., & Oki, M., “Technologies for targeting the peripheral pulmonary nodule including robotics”, Respirology, 2020, 25(9), 914-923, and NPL2 by Kato et al., “Robotized Catheter with Enhanced Distal Targeting for Peripheral Pulmonary Biopsy”, Published in: IEEE/ASME Transactions on Mechatronics (Volume: 26, pages 2451-2461, Issue: 5, October 2021).

Detection of peripheral pulmonary nodules are particularly challenging even when relying in robotic assisted technologies as described in NPL1 and NPL2. When the tip of a robotic bronchoscope approaches a peripheral pulmonary nodule, the physician aims the bronchoscope toward the nodule to take a sample. The targeted placement of a device (e.g., biopsy needle) is very important in these procedures. Targeting accuracy within millimeters is desired, especially if the target is small, or close to another organ, vessel, or nerve. However, due to the peripheral location of the nodule, the endoscopist has a tendency of “getting lost” in the peripheral airways. For this reason, physicians keep trying to aim a target (tumor or nodule) more accurately after they aim the catheter toward the target within an acceptable range. After various tries, a physician can end up with worse accuracy and has to go back to a previous location to try to realign the catheter tip with the intended target. This process results in suboptimal targeting and prolongs the procedure, which causes an increased mental burden for the physician and possible discomfort for the patient.

Therefore, there is a need for improved robotic catheter systems and methods for rapidly and accurately aligning the catheter tip with the intended target, displaying guiding information that can alleviate the user burden, reduce procedure time, and improve patient comfort.

### SUMMARY OF EXEMPLARY EMBODIMENTS

A system for, display controller connected to, and a method of, operating a robotic catheter system which is configured to manipulate a catheter having one or more bending segments along the catheter's length and a catheter tip at the distal end thereof, and which includes an actuator unit coupled to the bending segments via one or more drive wires arranged along a wall of the catheter. The method comprising: inserting at least part of the catheter into a bodily lumen along an insertion trajectory that spans from an insertion point to a target; causing the actuator unit to actuate at least one of the one or more drive wires to align the catheter tip with the target; determining the position and/or orientation of the catheter tip with respect to the target; and displaying information about an accuracy of alignment between the catheter tip with respect to the target.

According to an embodiment, an operator can refer to the history of the estimated accuracy of sampling, and can come back to the past posture of the robot using the history of the estimated accuracy of sampling. By displaying a history of estimated sampling locations, the operator can understand the best sampling location and the trend of the sampling among all attempts by referring to the objective information. Then, the operator can make their judgment whether they would like to execute one of the historical sampling locations or the current location or contusing targeting efficiently. This can prevent prolonging the procedure duration and suboptimal targeting. Also, since the operator does not need to remember the past sampling locations, it is possible reduce the operator's mental burden for the targeting.

It is to be understood that both the foregoing summary and the detailed description are exemplary and explanatory in nature and are intended to provide a complete understanding of the present disclosure without limiting the scope of the present disclosure. Additional objects, features, and advantages of the present disclosure will become apparent to those skilled in the art upon reading the following detailed description of exemplary embodiments, when taken in conjunction with the appended drawings, and provided claims.

### BRIEF DESCRIPTION OF DRAWINGS

FIG. 1 illustrates a robotic catheter system 1000 configured to manipulate a steerable catheter 104 having one or more bendable segments and a catheter tip at the distal end thereof, which can be used in an exemplary medical environment such as an operating room;

FIG. 2 illustrates a functional block diagram and components of the robotic catheter system 1000;

FIG. 3A illustrates a steerable catheter 104, according to one embodiment of the present disclosure. FIG. 3B and FIG. 3C illustrates principles of catheter tip manipulation by actuating one or more bending segments of the steerable catheter 104;

FIG. 4 illustrates a logical (data) block diagram of the robotic catheter system 1000, according to one embodiment of the present disclosure;

FIG. 5 illustrates components of a system controller 100 and/or a display controller 102;

FIG. 6 shows a planning operation 600 for steerable catheter 104, according to one embodiment of the present disclosure;

FIG. 7A shows a navigation workflow 700 according to an operation of the robotic catheter system 1000 to navigate the steerable catheter 104 to a target, and FIG. 7B illustrates a targeting workflow, according to one embodiment of the present disclosure;

FIG. 8 illustrates a virtual view of a targeting process as seen in a side-view image displayed on a main display 101-1 or secondary display 101-2, and FIG. 9 illustrates a virtual view of the targeting process, as seen in a first-person-view (FPV), according to an example of the present disclosure;

FIG. 10 illustrates a virtual view of a targeting process as seen in a side-view image displayed on a main display 101-1 or secondary display 101-2, and FIG. 11 illustrates a virtual view of the targeting process, as seen in FPV, according to another example of the present disclosure;

FIG. 12 illustrates a virtual view of a targeting process where a history of estimated sampling locations is displayed with time stamps, according to a further example of the present disclosure;

FIG. 13 illustrates a targeting process based on real-time FPV bronchoscopic images of branching structure, according to yet another example of the present disclosure; and

FIG. 14 illustrates a targeting process based on real-time fluoroscopic images of a branching structure 1410, according to another example of the present disclosure.

### DETAILED DESCRIPTION OF EXEMPLARY EMBODIMENTS

Aspects of the present disclosure can be understood by reading the following detailed description in light of the accompanying figures. It is noted that, in accordance with standard practice, the various features of the drawings are not drawn to scale and do not represent actual components. Several details such as dimensions of the various features may be arbitrarily increased or reduced for ease of illustration. In addition, reference numerals, labels and/or letters are repeated in the various examples to depict similar components and/or functionality. This repetition is for the purpose of simplicity and clarity and does not in itself limit the various embodiments and/or configurations the same components discussed.

Before the various embodiments are described in further detail, it shall be understood that the present disclosure is not limited to any particular embodiment. It is also to be understood that the terminology used herein is for the purpose of describing exemplary embodiments only, and is not intended to be limiting. Embodiments of the present disclosure may have many applications within the field of medical treatment or minimally invasive surgery (MIS).

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

As used herein, the term “real-time” is meant to describe processes or events communicated, shown, presented, etc. substantially at the same time as those processes or events actually occur. Real time refers to a level of computer responsiveness that a user senses as sufficiently immediate or that enables the computer to keep up with some external process. For example, in computer technology, the term real-time refers to the actual time during which something takes place and the computer may at least partly process the data in real time (as it comes in). As another example, in signal processing, “real-time” processing relates to a system in which input data is processed within milliseconds so that it is available virtually immediately as feedback, e.g., in a missile guidance, an airline booking system, or the stock market real-time quotes (RTQs).

The present disclosure generally relates to medical devices, and it exemplifies embodiments of an endoscope or catheter, and more particular to a steerable catheter controlled by a medical continuum robot (MCR). The embodiments of the endoscope or catheter and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute. In that regard, all directional references (e.g., upper, lower, upward, downward, left, tight, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure.

As used herein the term “catheter” generally refers to a flexible and thin tubular instrument made of medical grade material designed to be inserted through a narrow opening into a bodily lumen (e.g., an airway or a vessel) to perform a broad range of medical functions. The more specific term “steerable catheter” refers to a medical instrument comprising an elongated shaft made of one or more actuatable segments.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

In the present disclosure, the terms “optical fiber”, “fiber optic”, or simply “fiber” refers to an elongated, flexible, light conducting waveguide capable of conducting light from one end to another end due to the effect known as total internal reflection. The terms “light guiding component” or “waveguide” may also refer to, or may have the functionality of, an optical fiber. The term “fiber” may refer to one or more light conducting fibers.

### Robotic Catheter System

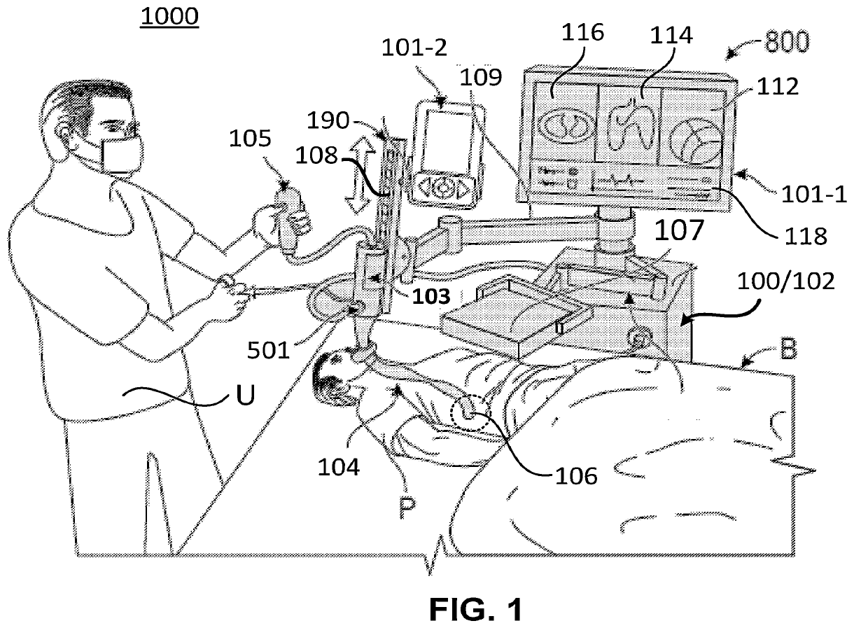

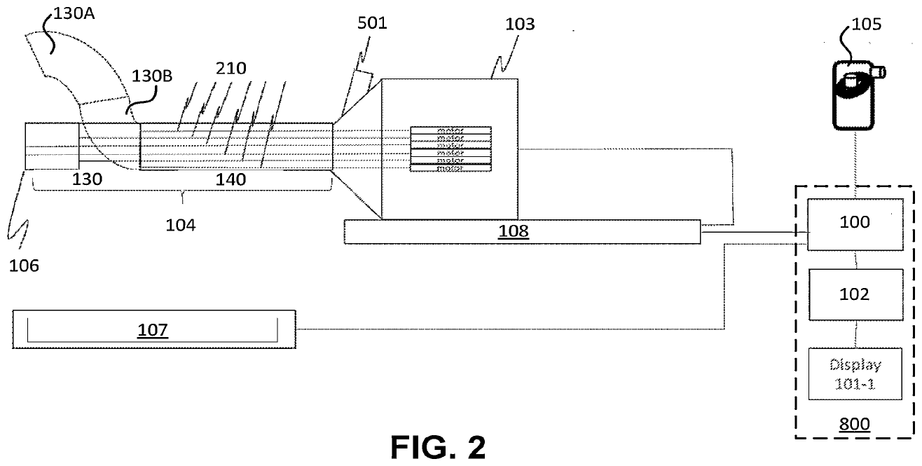

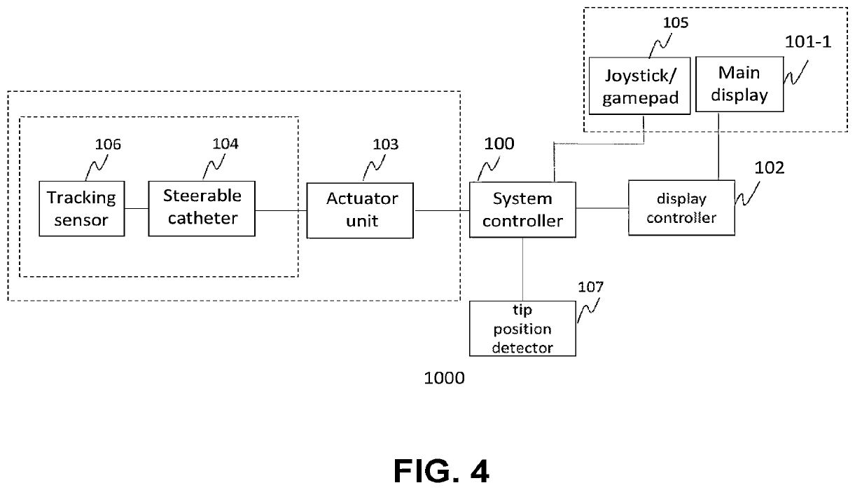

An embodiment of a robotic catheter system 1000 is described in reference to FIG. 1 through FIG. 4. FIG. 1 illustrates a simplified representation of a medical environment, such as an operating room, where a robotic catheter system 1000 can be used. FIG. 2 illustrates a functional block diagram of the robotic catheter system 1000. FIG. 4 illustrates a logical block diagram of the robotic catheter system 1000. In this example, the system 1000 includes a system console 800 (computer cart) operatively connected to a steerable catheter 104 via a robotic platform 190. The robotic platform 190 includes one or more than one robotic arm 109 and a linear translation stage 108.

A user U (e.g., a physician) controls the robotic catheter system 1000 via a user interface unit (operation unit) to perform an intraluminal procedure on a patient P positioned on an operating table B. The user interface may include at least one of a main display 101-1 (a first user interface unit), a secondary display 101-2 (a second user interface unit), and a handheld controller 105 (a third user interface unit). The main display 101-1 may include a large display screen attached to the system console 800 or mounted on a wall of the operating room. The secondary display 101-2 may include a compact (portable) display device configured to be removably attached to the robotic platform 190. Examples of the secondary display 101-2 include a portable tablet computer or a mobile communication device (a cellphone).

The steerable catheter 104 is actuated via an actuator unit 103. The actuator unit 103 is removably attached to the linear translation stage 108 of the robotic platform 190. The handheld controller 105 may include a gamepad controller with a joystick having shift levers and/or push buttons. In one embodiment, the actuator unit 103 is enclosed in a housing having a shape of a catheter handle. An access port 501 is provided in or around the catheter handle. The access port 501 is used for inserting and/or withdrawing end effector tools and/or fluids when performing an interventional procedure of the patient.

The system console 800 includes a system controller 100, a display controller 102, and the main display 101-1. The main display 101-1 may include a conventional display device such as a liquid crystal display (LCD), an OLED display, a QLED display or the like. The main display 101-1 provides a graphic interface unit (GUI) configured to display one or more of a live view image 112, an intraoperative image 114, and a preoperative image 116, and other procedural information 118. The preoperative image 116 may include pre-acquired 3D or 2D medical images of the patient acquired by conventional imaging modalities such as computer tomography (CT), magnetic resonance imaging (MRI), or ultrasound imaging. The intraoperative image 114 may include images used for image guided procedure such images may be acquired by fluoroscopy or CT imaging modalities. Intraoperative image 114 may be augmented, combined, or correlated with information obtained from a catheter tip position detector 107 and catheter tip tracking sensor 106. The catheter tip tracking sensor 106 may include an electromagnetic (EM) sensor, and the catheter tip position detector 107 may include an EM field generator operatively connected to the system controller 100. Suitable electromagnetic sensors for use with a steerable catheter are well-known and described, for example, in U.S. Pat. No.: 6,201,387 and international publication WO2020194212A1.

Similar to FIG. 1, the diagram of FIG. 2 illustrates the robotic catheter system 1000 includes the system controller 100 operatively connected to the display controller 102, to the actuator unit 103 via the robotic platform 190, and to the tip position detector 107. The tip position detector 107 is at a position spatially related to a three dimensional space where the catheter tip tracking sensor 106 operates. In this manner, the tip position detector 107 is able to track the functional position of the steerable catheter 104.

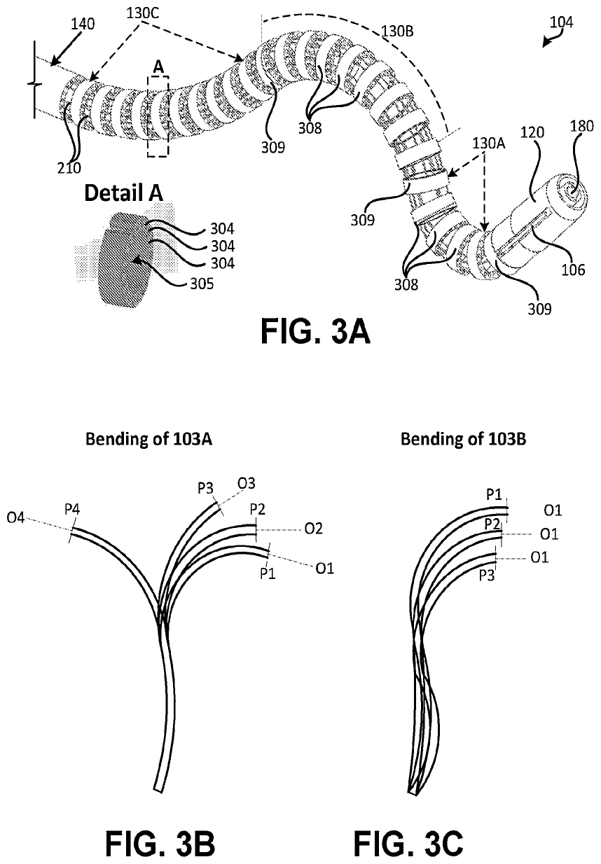

FIG. 3A shows an exemplary embodiment of a steerable catheter 104. The steerable catheter 104 includes a non-steerable proximal section 140, a steerable distal section 130, and a catheter tip 120. The proximal section 140 and distal section 130 are joined to each other by a plurality of drive wires 210 arranged along the wall of the catheter. The proximal section 140 is configured with thru-holes or grooves or conduits to pass drive wires 210 from the distal section 130 to the actuator unit 103. The distal section 130 is comprised of a plurality of bending segments including at least a distal segment 130A, a middle segment 130B, and a proximal segment 130C. Each bending segment is bent by actuation of at least some of the plurality of drive wires 210 (driving members). The posture of the catheter is supported by non-illustrated supporting wires (support members) also arranged along the wall of the catheter. The proximal ends of drive wires 210 are connected to individual actuators or motors of the actuator unit 103, while the distal ends of the drive wires 210 are selectively anchored to anchor members in the different bending segments of the distal section 130.

Each bending segment is formed by a plurality of ring-shaped components (rings) with thru-holes, grooves, or conduits along the wall of the rings. The ring-shaped components are defined as wire-guiding members 308 or anchor members 309 depending on their function within the catheter. Anchor members 309 are ring-shaped components onto which the distal end of one or more drive wires 210 are attached. Wire-guiding members 308 are ring-shaped components through which some drive wires 210 slide through (without being attached thereto).

Detail “A” in FIG. 3A illustrates an exemplary embodiment of a ring-shaped component (a wire-guiding member 308 or an anchor member 309). Each ring-shaped component includes a central opening which forms the tool channel 305, and plural conduits 304 (grooves, sub-channels, or thru-holes) arranged lengthwise equidistant from the central opening along the annular wall of each ring-shaped component. The non-steerable proximal section 140 is a flexible tubular shaft made of extruded polymer material. The tubular shaft of the proximal section 140 also has a central opening or tool channel 305 and plural conduits 304 along the wall of the shaft surrounding the tool channel 305. In this manner, at least one tool channel 305 formed inside the steerable catheter 104 provides passage for an imaging device and/or end effector tools from the insertion port 501 to the distal end of the steerable catheter 104.

An imaging device 180 that can be inserted through the tool channel 305 includes an endoscope camera (videoscope) along with illumination optics (e.g., optical fibers or LEDs). The illumination optics provides light to irradiate a lesion target which is a region of interest within the patient. End effector tools refer endoscopic surgical tools including clamps, graspers, scissors, staplers, ablation or biopsy needles, and other similar tools, which serve to manipulate body parts (organs or tumorous tissue) during examination or surgery.

The actuator unit 103 includes one or more servo motors or piezoelectric actuators. The actuator unit 103 bends one or more of the bending segments of the catheter by applying a pushing and/or pulling force to the drive wires 210. As shown in FIG. 3A, each of the three bendable segments of the steerable catheter 104 has a plurality of drive wires 210. If each bendable segment is actuated by three drive wires 210, the steerable catheter 104 has nine driving wires arranged along the wall of the catheter. Each bendable segment of the catheter is bent by the actuator unit 310 by pushing or pulling at least one of these nine drive wires 210. Force is applied to each individual drive wire in order to manipulate/steer the catheter to a desired pose. The actuator unit 103 assembled with steerable catheter 104 is mounted on the linear translation stage 108. Linear translation stage 108 includes a slider and a linear motor. In other words the linear translation stage 108 is motorized, and can be controlled by the system controller 100 to insert and remove the steerable catheter 104 to/from the patient's bodily lumen.

A tracking sensor 106 (e.g., an EM tracking sensor) is attached to the catheter tip 120. In this embodiment, steerable catheter 104 and the tracking sensor 106 can be tracked by the tip position detector 107. Specifically, the tip position detector 107 detects a position of the tracking sensor 106, and outputs the detected positional information to the system controller 100. The system controller 100 receives the positional information from the tip positon detector 107, and continuously records and displays the position of the steerable catheter 104 with respect to the patient's coordinate system. The system controller 100 controls the actuator unit 103 and the linear translation stage 108 in accordance with the manipulation commands input by the user U via one or more of the user interface units (the handheld controller 105, a GUI at the main display 101-1 or touchscreen buttons at the secondary display 101-2).

FIG. 3B and FIG. 3C show exemplary catheter tip manipulations by actuating one or more bending segments of the steerable catheter 104. As illustrated in FIG. 3B, manipulating only the most distal segment 103A of the steerable section changes the position and orientation of the catheter tip 120. On the other hand, manipulating one or more bending segments (103B or 103C) other than the most distal segment affects only the position of catheter tip 120, but does not affect the orientation of the catheter tip. In FIG. 3B, actuation of distal segment 103A changes the catheter tip from a position P1 having orientation O1, to a position P2 having orientation O2, to position P3 having orientation 03, to position P4 having orientation 04, etc. In FIG. 3C, actuation of the middle segment 103B changes the position of catheter tip 120 from a position P1 having orientation O1 to a position P2 and position P3 having the same orientation O1. Here, it should be appreciated by those skilled in the art that exemplary catheter tip manipulations shown in FIG. 3B and FIG. 3C can be performed during catheter navigation (i.e., while inserting the catheter through tortuous anatomies). In the present disclosure, the exemplary catheter tip manipulations shown in FIG. 3B and FIG. 3C apply namely to the targeting mode applied after the catheter tip has been navigated to a predetermined distance (a targeting distance) from the target.

The system controller 100 executes software programs and controls the display controller 102 to display a navigation screen (e.g., a live view image 112) on the main display 101-1 and/or the secondary display 101-2. The display controller 102 may include a graphics processing unit (GPU) or a video display controller (VDC). The display controller 102 generates a three dimensional (3D) model of an anatomical structure (for example a branching structure like the airway of a patient's lungs) based on preoperative or intraoperative images such as CT or MRI images, etc. Alternatively, the 3D model may be received by the system console from other device (e.g., a PACS sever). A two dimensional (2D) model can be used instead of 3D model. In this case, the display controller 102 may process (through segmentation) a preoperative 3D image to acquire slice images (2D images) of a patient's anatomy. The 2D or 3D model can be generated before catheter navigation starts. Alternatively, the 2D or 3D model can be generated in real-time (in parallel with the catheter navigation). In one embodiment, an example of generating a model of a branching structure is explained later. However, the model is not limited to a model of branching structure. For example, a model of a route direct to a target (a tumor or nodule or tumorous tissue) can be used instead of the branching structure. Alternatively a model of a broad space can be used for catheter navigation. The model of broad space can be a model of a place or a space where an observation or a task is performed by using the robotic catheter, as further explained below.

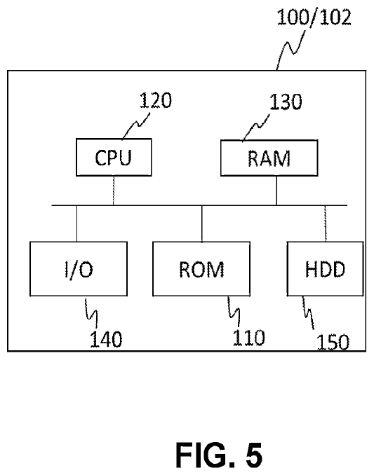

FIG. 5 illustrates components of the system controller 100 and/or the display controller 102. The system controller 100 and the display controller 102 can be configured separately. Alternatively, the system controller 100 and the display controller 102 can be configured as one device. In either case, the system controller 100 and the display controller 102 comprise substantially the same components. Specifically, the system controller 100 and display controller 102 may include a central processing unit (CPU 120) comprised of one or more processors (microprocessors), a random access memory (RAM 130) module, an input/output (I/O 140) interface, a read only memory (ROM 110), and data storage memory (e.g., a hard disk drive (HDD 150) or solid state drive (SSD)).

The ROM 110 and/or HDD 150 store the operating system (OS) software, and software programs necessary for executing the functions of the robotic catheter system 1000 as a whole. The RAM 130 is used as a workspace memory. The CPU 120 executes the software programs developed in the RAM 130. The I/O 140 inputs, for example, positional information to the display controller 102, and outputs information for displaying the navigation screen to the one or more displays (main display 101-1 and/or secondary display 101-2). In the embodiments descried below, the navigation screen is a graphical user interface (GUI) generated by a software program but, it may also be generated by firmware, or a combination of software and firmware.

The system controller 100 may control the steerable catheter 104 based on any known kinematic algorithms applicable to continuum or snake-like catheter robots. For example, the system controller controls the steerable catheter 104 based on an algorithm known as follow the leader (FTL) algorithm. By applying the FTL algorithm, the most distal segment 130A of the steerable section 130 is actively controlled with forward kinematic values, while the middle segment 130B and the proximal segment 130C (following sections) of the steerable catheter 104 move at a first position in the same way as the distal section moved at the first position or a second position near the first position.

The display controller 102 acquires position information of the steerable catheter 104 from system controller 100. Alternatively, the display controller 102 may acquire the position information directly from the tip position detector 107. The steerable catheter 104 may be a single-use or limited-use catheter device. In other words, the steerable catheter 104 can be attachable to, and detachable from, the actuator unit 103 to be disposable.

During a procedure, the display controller 102 generates and outputs a live-view image or a navigation screen to the main display 101-1 and/or the secondary display 101-2 based on the 3D model of a patient's anatomy (a branching structure) and the position information of at least a portion of the catheter (e.g., position of the catheter tip 120) by executing pre-programmed software routines. The navigation screen indicates a current position of at least the catheter tip 120 on the 3D model. By observing the navigation screen, a user can recognize the current position of the steerable catheter 104 in the branching structure. Upon completing navigation to a desired target, one or more end effector tools can be inserted through the access port 501 at the proximal end of the catheter, and such tools can be guided through the tool channel 305 of the catheter body to perform an intraluminal procedure from the distal end of the catheter.

The tool may be a medical tool such as an endoscope camera, forceps, a needle or other biopsy or ablation tools. In one embodiment, the tool may be described as an operation tool or working tool. The working tool is inserted or removed through the working tool access port 501. In the embodiments below, an embodiment of using a steerable catheter to guide a tool to a target is explained. The tool may include an endoscope camera or an end effector tool, which can be guided through a steerable catheter under the same principles. In a procedure there is usually a planning procedure, a registration procedure, a targeting procedure, and an operation procedure.

### Planning Procedure

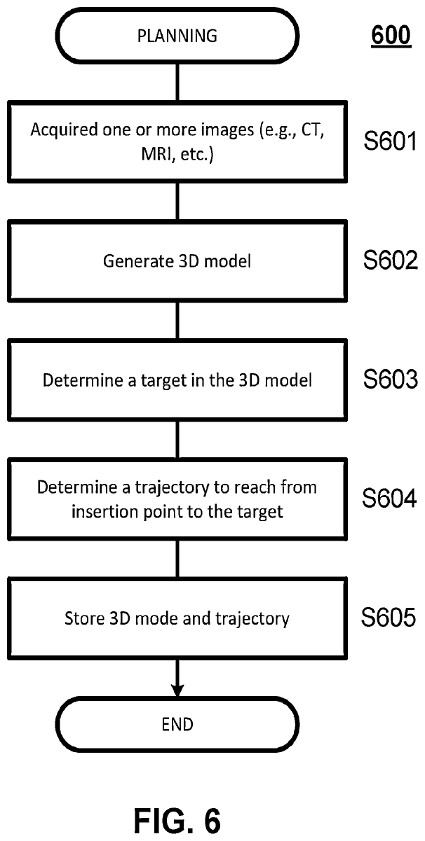

FIG. 6 shows a planning procedure 600 for planning the insertion of steerable catheter 104. These steps are performed by the system controller 100 executing a software program read from the ROM 110 or HDD 150 by CPU 120. In step S601, medical images of the patient, such as CT or MRI images of the patient, are acquired. In step S602, a three dimensional model of an anatomy like a branching structure (for example, an airway model of lungs) is generated based on the acquired images. In step S603, a target on the branching structure is determined based on a user input. For example, the user selects a target on the branching structure by using a pointer (e.g., by clicking a mouse) or by manually touching the screen of main-display 101-1 or secondary display 101-2. In step S604, a trajectory of the steerable catheter 104 to reach the target on the branching structure is determined by CPU 120 based on the user selection and a user instruction. In step S604, the user instruction can include the marking of an insertion point, and the marking of one or more points along the branching structure between the insertion point and the target. In step S605, the generated three dimensional model and decided trajectory on the model is stored in the RAM 130 or HDD 150. In this manner, a 3D model of a branching structure is generated. Also, a location of the target, and the trajectory on the 3D model, and physical parameters of the catheter (catheter length, diameter, torsion and bending limits, etc.) can be stored before navigation of the steerable catheter 104 is started.

### Registration Procedure

At the beginning of the operation of steerable catheter 104, system 1000 will perform a 3D model-to-robot registration to calculate transformation of coordinates of robotic catheter system into coordinates of the 3D model. For example, the registration can be executed with a known method like a point-set registration with fiducial markers in the 3D model by measuring the positions of fiducial markers on the patient by using EM tracking sensor 106. The registration process may include catheter-to-patient registration or device-to-image registration where registration of catheter coordinates to coordinates of a tracking system can be performed in any known procedure. Examples of the registration process are described in U.S. Pat. Nos.: 10,898,057 and 10,624,701, which are hereby incorporated by reference herein for all purposes. After the registration, system 1000 can provide to the system controller 100 and/or to the display controller 102, the 3D model of the branching structure, the target, the route from the insertion point to the target, and the current position and orientation (pose) of the distal tip. We call this view an EM virtual view in the rest of this manuscript.

### Navigation Procedure

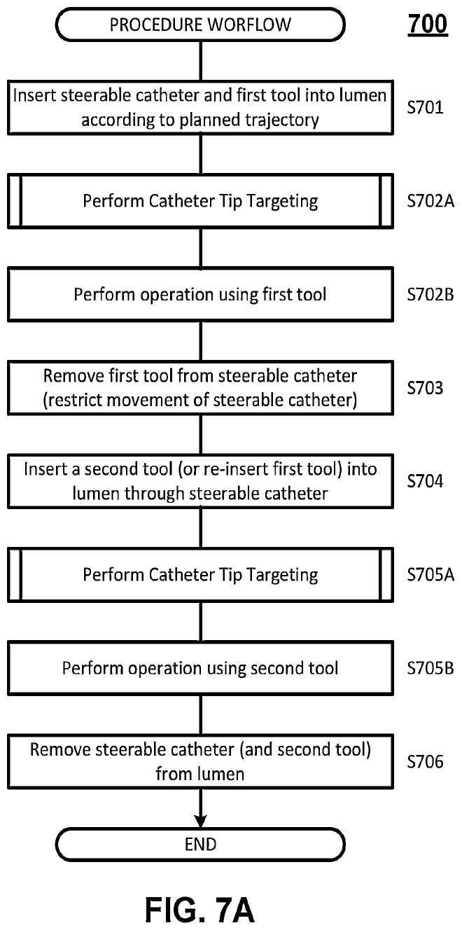

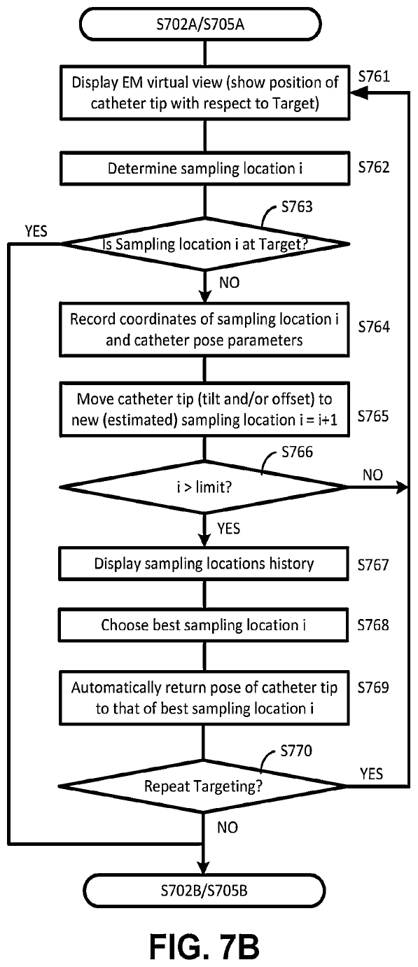

FIG. 7A shows a navigation workflow 700. In this embodiment, the navigation workflow shows an operation of the robotic catheter system 1000 to navigate the steerable catheter 104 and at least one tool through a bodily lumen of a branching structure. The at least one tool can be inserted into and/or removed from the steerable catheter 104. These steps are performed by executing a software program read from the ROM 110 or HDD 150 by CPU 120 of the system controller 100 or the display controller 102.

At step S701, the steerable catheter 104 and a first tool (for example, an endoscope camera) is inserted into a branching structure (for example, an airway of a patient) in accordance with the plan defined by the planning procedure 600. Specifically, at step S701, the actuator unit 103 is mounted onto the linear translation stage 108 of robot platform 190; a sterile catheter is attached to the actuator unit 103 (catheter handle); and the assembled robotic catheter is aligned with an insertion point of the patient P. The insertion point can be a natural orifice or a surgically created one. The robot platform 190 proceeds to move the steerable catheter 104 from the insertion point into the branching structure. At the same time, by operating the handheld controller 105, the user (for example a physician) sends input signals to the system controller 100, which in turn controls the actuator unit 103 to apply pushing or pulling forces to selected drive wires 210. As the drive wires 210 move in response to the applied force, the pushing or pulling force bends the one or more bending segments of steerable catheter 104 to navigate through the branching structure until the catheter tip 120 reaches the intended target.

The steerable catheter 104 and the first tool can be inserted into the branching structure independently or at the same time, depending on the type of tool being used and the type of procedure being performed. For example, insertion of the steerable catheter 104 independently of the first tool may be necessary or advantageous in certain circumstances. For easier handling, the steerable catheter 104 can be inserted without a tool through an endo-tracheal tube (ETT) until a desired location, and then a tool is inserted through the tool channel 305. On the other hand, the steerable catheter already assembled with the first tool can be inserted into the branching structure at the same time when the steerable catheter is assembled with an endoscope camera (a videoscope). In this case, the endoscope camera is set in the catheter tip 120, and the catheter with endoscope camera are inserted into the branching structure (airway of a lung) of a patient to reach a predetermined target (e.g., a nodule in the lung). A physician can control the posture of the catheter by operating the handheld controller 105 during catheter insertion, while the endoscope camera acquires a live view image of the branching structure. A captured image (a static image or a moving image) captured by the endoscope camera is displayed on the one or more displays (a main display 101-1 and/or a secondary display 101-2).

By observing the displayed image, the physician can determine the posture of the catheter and more accurately guide the catheter tip to the intended target. More specifically, after guiding the catheter tip to a depth sufficiently near the intended target, the robotic platform 109 stops insertion of the catheter (stops navigation mode). Subsequently, at step S702A, the system enters a targeting mode, and the user performs a targeting process, as explained below with reference to FIG. 7B.

At step S702B, after the catheter tip 120 and the first tool are properly aligned with the target, an operation by the first tool may be performed. The operation of the first tool at the target is not limited to an actual procedure. For example, in the case where the first tool is an endoscope camera, the endoscope camera may capture a static or moving image of the target (e.g., an image of a “nidus” buildup within an airway of a lung). In a case where the first tool is a biopsy tool such as a needle or forceps, the operation of the first tool may include sampling of a tissue at the target location. Alternatively, in a case where the first tool is an endoscope camera, the endoscope camera may be used only for capturing images of the bodily lumen along the trajectory from an insertion point to the target. In this case, the system may record any particular maneuver of the catheter or operation of the endoscope camera other than capturing images, as the catheter and endoscope camera advance through the bodily lumen.

At step s703, after the operation with the first tool, a tool removal process is performed. More specifically, at S703, the first tool is removed from the steerable catheter 104. When the removal of the first tool is detected, the movement of the steerable catheter 104 is automatically locked. To maintain alignment of the catheter tip to the target (i.e., to maintain the pose of the catheter), movement of the steerable catheter 104 is restricted automatically by the system controller 100. For example, the linear translation stage 108 and the handheld controller 105 are locked so that the endoscope camera can be removed from the catheter without changing the pose of the catheter. In this manner, positional relationship between the target and the catheter tip can remain substantially unchanged during the removal of the endoscope camera from the catheter.

At step S704, a second tool can be inserted (or the first tool is re-inserted) into the steerable catheter 104. For example, in the case where the first tool was an endoscope camera, the second tool may be a biopsy tool (or an ablation needle), which now is inserted into the steerable catheter 104 after the endoscope camera (first tool) was removed from the catheter. Here, it will be understood that during withdrawal of the first tool and insertion of the second tool, there is a possibility of some movement of the catheter and/or the patient which may cause a change in the pose of the catheter.

Therefore, at step S705A, after the second tool is inserted (or after the first tool is re-inserted) into the steerable catheter 104, the system again enters a targeting mode, and the user confirms or performs a targeting process, as explained below with reference to FIG. 7B.

At step S705B, after the targeting process, an operation of the inserted second tool (or re-inserted first tool) is performed. For example, in the case that the first tool is an endoscope camera and the second tool is a biopsy or ablation tool, a biopsy operation or an ablation procedure is performed by the second tool at step S705B. In addition, it will be understood that more than one operation may be performed with the second tool. For example, in the case that the second tool is a biopsy or ablation tool, several samplings might be necessary for a biopsy operation or plural ablations may be necessary to fully treat a large tumor.

At step S706, after the operation of using the second tool is completed, the second tool and the steerable catheter 104 are removed from the bodily lumen or branching structure. At step S706, the second tool can be removed together with the steerable catheter 104, or the second tool can be removed before the steerable catheter 104. It should be naturally understood that the process of FIG. 7A is not limited to the operations of first and second tools, as any number of tools can be used under the same principles disclosed herein.

### Targeting Procedure

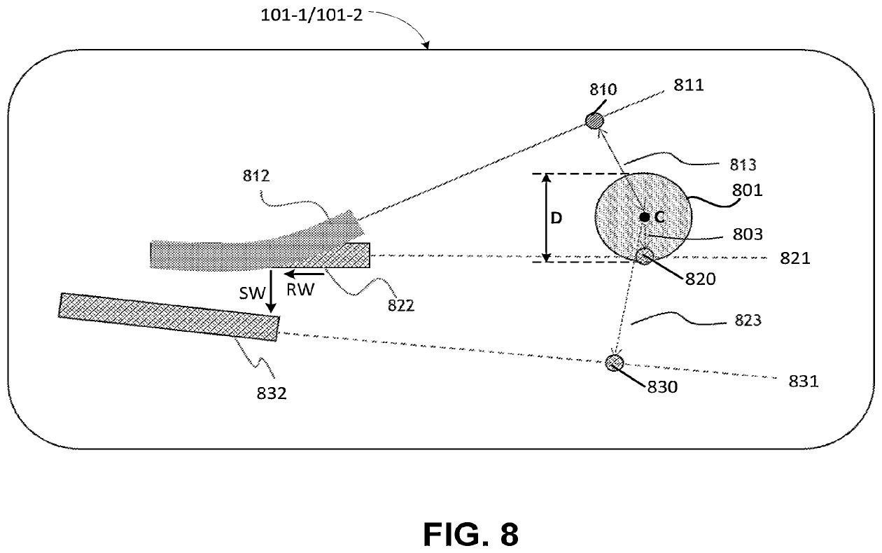

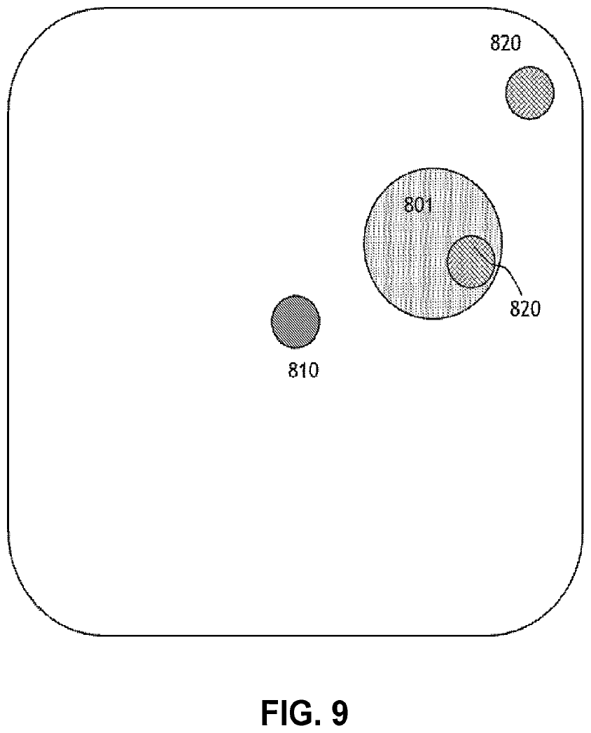

FIG. 7B illustrates a targeting workflow for the targeting process of steps S702A and/or S705A, according to an embodiment of the present disclosure. In the targeting process, at step S761, the display controller 102 causes the main display 101-1 or secondary display 101-2 to display the current position of the catheter tip 120 with respect to the target in an EM virtual view. FIG. 8 is an example of an EM virtual view showing a plurality of postures of the catheter tip aligned consecutively with a plurality of sampling locations in or around a target 801. A first posture 812 shows the catheter tip in an orientation 811, a second posture 822 shows the catheter tip in an orientation 821, and a third posture 832 shows the catheter tip in an orientation 831. At step S762, from the EM virtual view, the user observes and determines what sampling location can be reached from the current pose (position and orientation) of catheter tip 120 (i.e., the user determines a sampling location i at or near the target 801 which can be reached from the catheter tip). Therefore, at step S763, the system or the user determines if the sampling location i is at the target. If the catheter tip is already well aligned with the target, such that the sampling location i is at or close to the center C of the target (YES at S763), the process advances to step S702B or S705B (in FIG. 7A), where the intended operation with the first or second tool can be performed.

If the sampling location is not at the target (NO at S763), the process advances to step S764. At S764, the system records the coordinates of the sampling location i, and records the parameters of the steerable catheter 104 (e.g., pose (position and orientation)) with respect the sampling location i and or with respect to the center of target 801. For example, the system records in memory (HDD 150) the position and orientation (pose or posture) of the catheter tip and the coordinates of the sampling location i with respect to the target. The system also adds a marker (displays an icon) corresponding to the coordinates of the sampling location i with respect to the target. For example, in FIG. 8, the system records the first posture 812 of the catheter tip 120, adds a marker 810 as the first sampling location i, and records coordinates of the marker 810 at a distance 813 with respect to the target 801. In this example, the system records the first distance 813 between the center C of target 801 and the marker 810 as the first distance to the first sampling location. Here, the system may link (a) the data corresponding to the commands used to place the catheter tip in the first posture 812, (b) the data corresponding to the coordinates of the marker 810, and (c) the data of the first distance 813.

At step S765, the user manipulates the catheter tip 120 with the handled controller 105 to tilt and/or offset the catheter tip towards a new sampling location (a sampling location i=i+1). The tilting and/or offsetting of the catheter tip is done with the intention of better aligning the catheter tip with the target (hence the name “targeting”). For example, in FIG. 8, the user may control the most distal bending segment of the steerable catheter 104 to bend the catheter tip 120 from the first posture 812 to a second posture 822. The result of step S765 is that the catheter tip becomes realigned with respect to the target 801. Therefore, in the second posture 822, the catheter tip is now oriented in a direction along the edge of target 801. Here, the system or the user adds a second marker 820 indicative of a second estimated sampling location (sampling location i=i+1). When the second marker is added, the system records the coordinates of the second marker and the distance 803 from the second marker 820 to the center C of the target 801. After the catheter tip is realigned from the first posture 812 to a second posture 822, the process returns to step S761, where the display controller 102 now displays in the virtual view the first and second markers 810 and 820 respectively indicative of the first and second estimated positions. At S763, the system again determines if the currently estimated sampling location is at the target.

If the system or the user considers that the second sampling location indicated by the second marker 820 is not “at the target”. For example, if the user considers that the catheter tip can be further realigned to obtain a better sampling location, the user will again use the gamepad controller to bend one or more of the bending segments and thereby again move the catheter tip at step S765. For example, referring again to FIG. 8, if the second sampling location 820 is located further than a minimum distance 803 from the target center C, the user may again control one or more of the bending segments to move the catheter tip. Here, the user may control one or more bending segments other than the most distal bending segment of the steerable catheter 104 to offset the catheter tip 120 in backward direction (RW) and/or in a sideways direction (SW) to place the catheter in a third posture 832. Here, the system again records the commands used to place the catheter tip 120 in the third posture 832, and adds a third marker 830 corresponding to the estimated sampling location (third sampling location). The system may also record the coordinates of the third marker 830 and a distance 823 representative of how much the orientation 831 had diverted away from the target 801.

The foregoing targeting process can be performed iteratively a predetermined number of times until the sampling location is at the target, or until a limit of iterations have occurred. Therefore, at step S766, the system or the user determines if a number of sampling locations i greater than a predetermined limit have been processed. For example, the limit of iterations can be determined based on the parameters (location, size, shape, etc.,) of the target. When the limit of iterations has been reached, the process advances to step S767.

At S767, the system displays all of the sampling locations (a sampling locations history). At step S768, from the displayed sampling locations history, the user can now choose the best sampling location i (e.g., the sampling location nearest to the target). In FIG. 8, the system or user may decide that the sampling location defined by marker 820 is the sampling location nearest to the center C of the target 801, and therefore is the best sampling location of all estimated sampling locations. At step S769, in response to the user selecting the marker 820, the system automatically returns the catheter tip to the pose corresponding to the chosen sampling location. More specifically, at S769, the system refers back to the recorded coordinates (position and orientation) and to the commands (tilting and/or offsetting) used when the catheter tip was in the second posture 822. To this end, as noted above, it is advantageous that the system is configured to record and store (a) the data corresponding to the commands used to place the catheter tip in a given posture, (b) the data corresponding to the coordinates of the marker assigned to the given posture, and (c) the data of the level of targeting accuracy (e.g., the distance) with reference to the target. The system may store these data in a hyperlinked manner such that, when the user selects a maker of higher accuracy, the system automatically places the catheter tip in the posture corresponding to the selected marker. In this manner, the user can select a targeting path with the highest accuracy, and the display device assists the user with carrying out the treatment procedure by providing visual targeting assistance and image overlay. It is contemplated that targeting paths to various regions of interest within the target can be defined, for example, when necessary to avoid obstacles, such as critical structures during ablation or sampling. Targeting accuracy can be defined as the Euclidean distance between the catheter tip (which hold the ablation probe) and the center of target (tumor center). In this regard, it is also contemplated that the targeting procedure can also address scenarios where there is movement of internal organs or shifting of the targeting trajectory due to movement of the patient's body.

At step S770, the user may now decide if to repeat the targeting process (YES at S770) or proceed to perform the desired operation at S702B or 705B (NO at S770).

FIG. 8 through FIG. 14 illustrates various examples of displaying a virtual view during a targeting procedure, according to the present disclosure. FIG. 8 provides a graphic illustration (a virtual view) of the various postures of the catheter tip and the corresponding estimated sampling locations with respect to the target 801, as explained above. FIG. 9 shows a virtual view of estimated sampling locations indicated by the first marker 810, the second marker 820, and the third marker 830 with respect to the target 801. The virtual view of FIG. 9 represents a view as seen in a first-person-view (FPV) from the catheter tip 120, according to a first embodiment of the present disclosure. As used herein, the concept of FPV refers to a method of controlling (navigating) the steerable catheter as if the eyes of the operator are in catheter tip (i.e., as seen from the user's point of view, if the eyes of the user were in the tip of the catheter).

In this embodiment, the steerable catheter 104 is first navigated through the branching structure to a point where the catheter tip 120 is close to a target. This process is referred to as a navigation mode in which the operator bends only the most distal section of the catheter by commanding the actuator unit 103 with the handheld controller 105. The rest of catheter sections are controlled by a FTL algorithm when the catheter is moved forward. When FTL navigation is implemented correctly, the steerable catheter 104 can follow the branching structure (e.g., airways of a lung) with minimal interaction to the wall of the bodily lumen (e.g., airway walls).

After the catheter tip 120 reaches close to the target, the operator switches the mode from the navigation mode to a targeting mode. The targeting mode, as explained above with reference to FIG. 7B, allows the operator to bend one or more bending segments of the catheter but not insert or remove the steerable catheter 104 (i.e., without advancing or withdrawing the catheter). The targeting process is necessary to determine the optimal position and orientation (optimal pose) of the catheter tip for performing a desired procedure (e.g., sampling or ablating) on the target.

According to this embodiment, the system provides two different bending operations for targeting. The first bending operation is tilting (changing orientation) of the catheter tip 120. For tilting the catheter tip, the operator bends only the most distal segment of the catheter by inputting commands with the handheld controller 105 and causing the actuator unit 103 to selectively apply a force to drive wires 210 attached to the most distal segment of the catheter (see FIG. 3B). The second bending operation is offsetting of the catheter tip 120. For offsetting the catheter tip 120, the operator can move laterally the catheter tip position while keeping the orientation (tilt) of the catheter tip. Here, the movement necessary for offsetting the catheter tip can be achieved by controlling one or more of the bending segments other than the most distal segment (see FIB. 3C). To that end, the system controller 100 will generate appropriate commands for the actuator unit 103 to control each of the bending segments individually by using the kinematics of the steerable catheter 104. By combining these two operations (tilting and offsetting), for example, the operator can aim the catheter tip to multiple locations in or around the target area with the same orientation.

During the targeting mode, the current position and orientation of the catheter tip 120 with respect to the target are graphically shown in a virtual view, in the main display 101-1 and/or the secondary display 101-2. The virtual view of the catheter tip and the target can be shown in different view angles. For example, FIG. 8 is an example of a side view, while FIG. 9 is an example of a first-person view (FPV). The target 801 can be shown with at least two representative features, which are the target's diameter (D) and its center position (C). When the target is not spherical, any other dimension (e.g., length or width or an approximate thereof) can be used as a parameter along with the center position.

The system controller 100 computes, and the display controller 102 displays, the sampling locations based on the position and orientation of the catheter tip obtained from the tip position detector 107 (e.g., an EM tracking system). While there are multiple approaches to define the sampling locations (810, 820, 830), in this embodiment, an optimal or best sampling location can be defined as the closest point on the distal tip orientation from the center C of the target.

The system controller 100 also computes, and the display controller 102 displays, the history of sampling locations. Since display controller 102 displays each real-time estimated sampling location, and stores these sampling locations as historical locations in association with the history of input commands defining the posture of the catheter, the system controller 100 can refer back to the previously displayed sampling locations. By showing the historical estimated sampling locations, the operator can compare the current location with historical estimated sampling locations, and understand the targeting tendency effectively and intuitively. For example, the operator can understand the best (closest) sampling location among all attempts executed to a certain point in time, or the targeting trend (approaching to target, or deviating from target) during manipulation of the steerable catheter 104. This interactive and visual process helps the operator make a decision about an end point of the targeting step with objective information. After making a decision about the best estimated pose for performing a procedure, the operator can control the catheter to recreate the posture for one of the recorded sampling locations using the input history of the commands stored by the system. To accomplish this, the user simply selects the marker indicative of highest targeting accuracy, and the system automatically recreates the catheter posture based on the stored positional history. In this manner, is possible to recreate a past posture of the catheter using the visual information on the display and the input the history of the commands. Incidentally, this targeting process can increase targeting accuracy, reduce duration of the procedure as whole, as well as reduce the user's mental burden. Moreover, as noted above, it is possible to also cover scenarios where the “history” that the system is re-creating can be modifiable by the user. For example, if halfway through the automatic re-insertion the clinician realizes that the body has moved and an organ has shifted, and the path it is attempting to retrace will collide with a wall or sensitive tissue, the clinician can enter movement commands to the catheter to align the catheter tip with the proper direction, and the software will update the history accordingly. For example, the controller recreates the posture of the catheter using the history of input commands modified or replaced by the input commands input by the clinician. Then the system can continue automatic re-insertion incorporating those modifications.

The virtual view can be displayed in several ways such as a first-person view as illustrated in FIG. 9 or a perspective view, or a side-view as shown in FIG. 8. The first-person view is a view from the distal end of the catheter tip. In the FPV, the current orientation of the catheter tip is represented by the marker 810 at the center of view. Also, in the FPV, the current estimated sampling location is at the same center of the viewing display. That is, in the FPV, the center marker 810 is associated with the current orientation of the catheter tip and the direction of the sampling location. The estimated sampling location history is shown by the second marker 820, and the third marker 830. It should be understood that several more makers associated with estimated sampling locations can be displayed. However, to ease the mental burden of the user, it can be advantageous to display makers corresponding to only the closest points of the estimated sampling locations. Furthermore, in the FPV, it may be advantageous to show at the center of the view the real-time normal vector of the catheter tip.

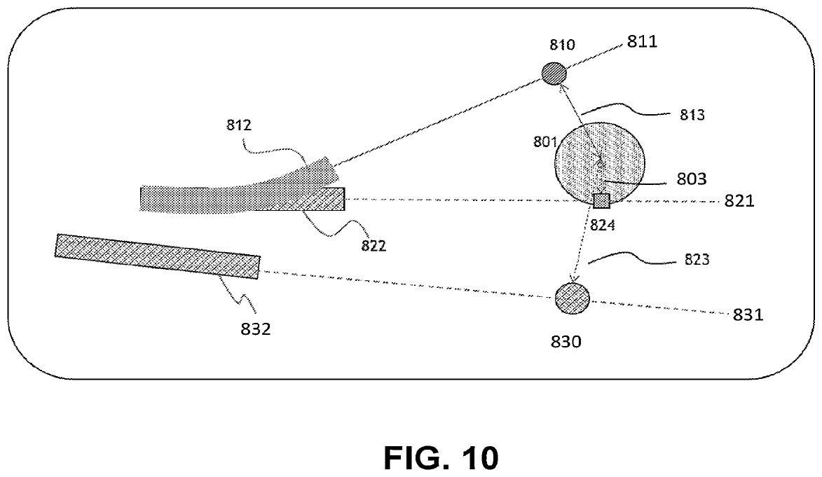

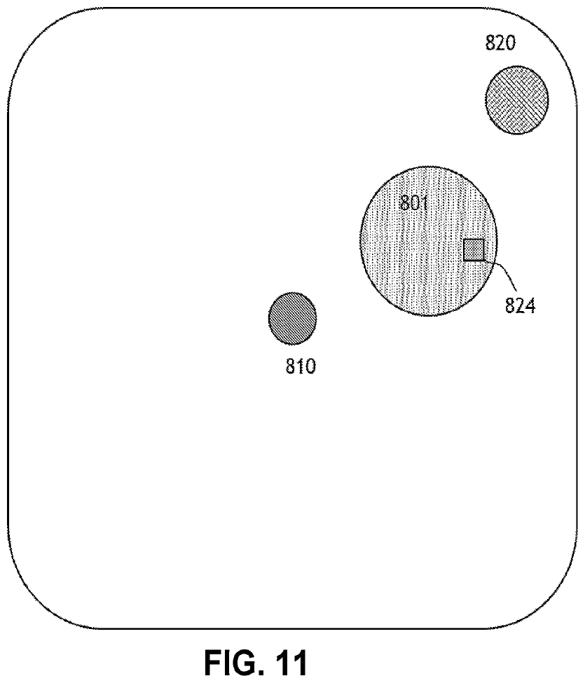

FIG. 10 illustrates a virtual view of a targeting process as seen in a side-view image displayed on a main display 101-1 or secondary display 101-2, and FIG. 11 illustrates a virtual view of the targeting process, as seen in FPV, according to a second embodiment of the present disclosure. In this embodiment, besides all features of the first embodiment, the virtual view shows a level of accuracy for the estimated sampling locations. The level accuracy can be defined as the distance between the center C of the target 801 and the position (coordinates) of sampling locations defined by the first marker 810, the second marker 820, and the third marker 830.

In this embodiment, the marker corresponding to the location of best targeting accuracy among all historical attempts can be shown as a different shape. For example, in FIG. 10 and FIG. 11 the best location is shown as a square marker 824. Also, the size of the marker can represent the magnitude (level) of the accuracy. In FIG. 10 and FIG. 11, the smaller the size of marker 824 equates to the better accuracy of targeting. According to this embodiment, an operator can easily decide the best estimated location of sampling using the information about accuracy and the distance between the direction (orientation 821) of the catheter tip and the center of the target.

In at least one embodiment, if the targeting process does not reach the desired level accuracy, the operator can exit the targeting mode and reverse to the navigation mode. Once the system returns to the navigation mode, the operator can move the catheter forward or backward (closer to or farther from the target). Then the operator can return to the targeting mode after moving the catheter tip to a better location forward/backward.

As described with reference to FIG. 7B, a new marker corresponding to a closest point can be added and displayed after every completion of a series of input commands sent to the robot defining the posture of the robot, or when the new point is more than 1 mm away from the previous points, or every predetermined time of targeting (e.g., every 5 seconds) according to the operator's choice.

In this regard, according to the embodiment of FIG. 10 and FIG. 11, an operator can also refer to the history of accuracy of estimated sampling locations. In such embodiment, all accuracy calculated in the past targeting attempts are stored by the system controller 100 in memory HDD 150 (or ROM 110) in association with the input history of the commands defining the posture of the catheter. When an operator clicks one of the markers representing the history of the sampling locations shown in the virtual view, the system controller 100 picks up the corresponding posture of the steerable catheter from the stored data, and sends a corresponding command to actuator unit 103 to set the steerable catheter 104 to the corresponding posture. According to this embodiment, an operator can come back to the past posture of the catheter by only clicking a marker of an estimated sampling location shown in the virtual view.

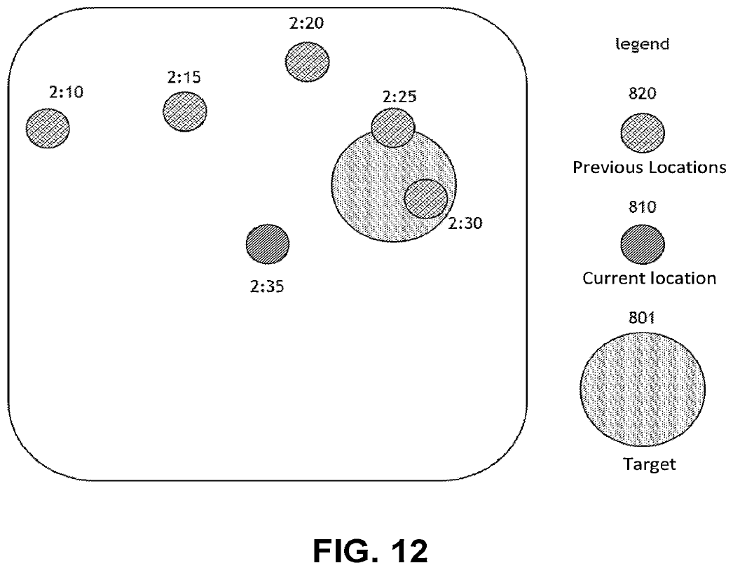

FIG. 12 illustrates a virtual view of a targeting process where a history of estimated sampling locations is displayed with time stamps, according to another embodiment of the present disclosure. In this embodiment, all accuracy calculated in the past targeting attempts are stored by the system controller 100 in association with the history of input commands defining the posture of the catheter. In addition, parameters of a secondary modality can be recorded by the system controller 100 to ensure the pose of the catheter and control commands are accurately tracked. In one embodiment, the system may use a respiratory phase of the patient's breathing cycle (e.g., defined by a ventilator) to track targeting attempts of the catheter. In this case, the system can use the ventilator to send a respiratory phase to the storage device during targeting. Then, the controller sends input commands to the robotic catheter to recreate the posture at the same respiratory phase when an expected position of the sampling is selected.

In FIG. 12, to avoid potential system hysteresis (lagging of an effect behind its cause), e.g., due to the patient's breathing cycle, a history of estimated sampling locations during a targeting process can be stored with time stamps of the respiratory phase (e.g., the inspiration phase, the expiration phase, or both). In this case, an operator may choose markers (points) corresponding to timed events of the targeting process to determine the most accurately estimated sampling location. For example, in FIG. 12, the operator may choose two markers shown in the virtual view with a time stamp corresponding to the start and end events of a targeting attempt. In response to the user selecting the two markers, the system controller 100 sends a command to the actuator unit 103 to recreate the posture of the catheter at the starting point using the history of input commands stored. After the robot recreates the posture of catheter at the starting point, the system controller waits until the end point of the respiratory phase (e.g., end of inspiration phase or end of expiration phase) to start moving the catheter tip towards the target. After the system controller confirms that the start and end points of the stored time stamps match the respiratory phase, the controller sends the same history of input commands to the actuator, and stops the catheter movement when the robot reaches the end point. According to this embodiment, an operator can avoid potential issues caused by delays or unsynchronized breathing motion during a targeting process. The system controller should be synchronized with the respiratory cycle of a patient. In this manner the system can record the input commands with reference to the respiratory phase of the patient. When the user requests the system to recreate a posture of the catheter, the system should send the input signal to the actuator at the same respiratory phase as that in which the input signal was recorded during targeting.

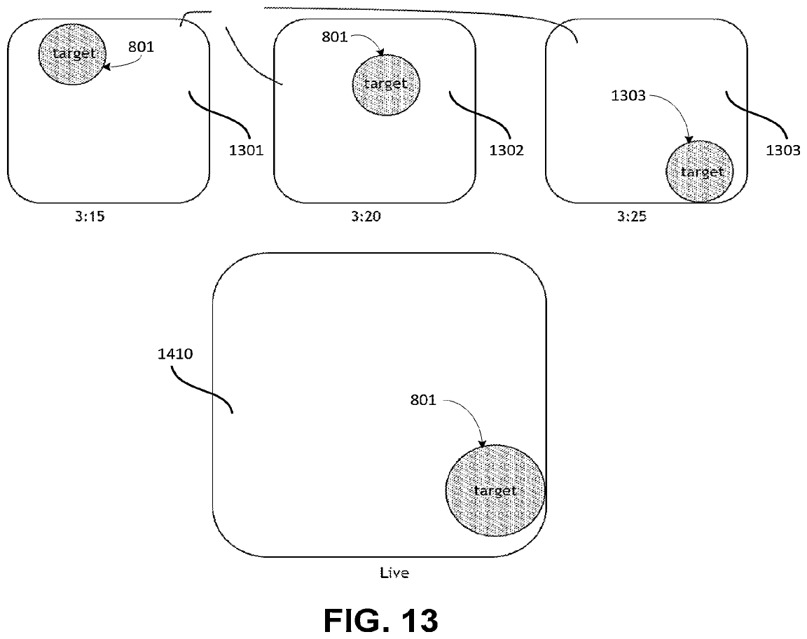

FIG. 13 illustrates a targeting process based on real-time FPV bronchoscopic images of a branching structure, according to another embodiment of the present disclosure. In this embodiment, a bronchoscopic camera captures an image of the branching structure every predetermined amount of time (e.g., very 5 seconds) during the targeting process, and the corresponding input commands defining the posture of the catheter tip to acquire each image is stored by the system controller 100 at the time when each of the images is captured. The previously captured images include a first image 1301, a second image 1302, and a third image 1303 each of which is stored and displayed with a respective time stamp. A currently captured image (a live view image) corresponding to the current position and orientation (current pose) of the catheter tip is also shown in the main display 101-1 and/or the secondary display 101-2. Here, bronchoscopic images 1301, 1302 and 1303 with older time stamps can be shown in smaller size than a real-time bronchoscopic image 1410 showing the current position of the catheter tip 120 with respect to the target 801. When an operator selects one of previously captured images showing the best alignment between the catheter tip and target, the stored corresponding input command or commands is/are sent to the actuator unit 103 to recreate the posture of the catheter at the time when the image was captured. According to this embodiment, an operator can come back to a past posture of the catheter by playing the series of captured images, and selecting a past captured image showing the estimated sampling location. In FIG. 13, the operator may chose the second image 1302 as the image representing the best estimated sampling location because this image shows the best alignment between the catheter tip and target 801.

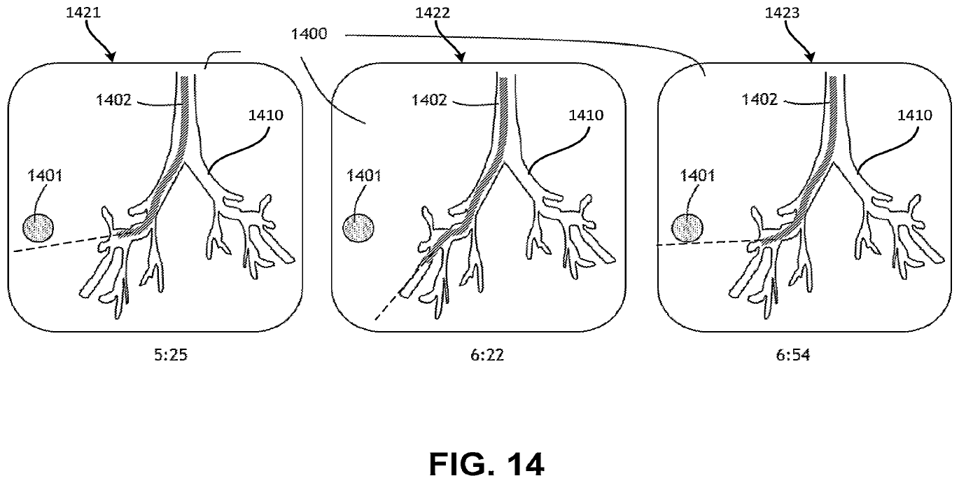

FIG. 14 illustrates a targeting process based on real-time fluoroscopic images of a branching structure 1410, according to another example of the present disclosure. FIG. 14 illustrates a process where the system receives time-stamped real-time fluoroscopic images of a branching structure 1410 during a targeting process. In this embodiment, as a user pushes a button of the handheld controller 105 during the targeting process, a corresponding input command defining the posture of the catheter is represented by a trajectory 1402; and this input command is stored in the system's memory by the system controller 100 in association with the process of capturing the fluoroscopic images. In this embodiment, a first fluoroscopic image 1421 is captured at time stamp 5:25 when an operator pushes a button of the handheld controller 105 during an initial targeting step when the catheter tip is close to being aligned with a target 1401. Here, the corresponding input command defining the posture (for trajectory 1402) of the catheter is stored in memory by the system controller 100 when the first fluoroscopic image 1421 is captured. Since the catheter tip is not fully aligned with the target 1401, the user may try a different targeting trajectory. A second fluoroscopic image 1422 is captured at time stamp 6:22 when the operator pushes a button of the handheld controller 105 in the middle of the targeting process when the catheter tip has shifted away from target 1401. Here, the corresponding input command defining the posture (for trajectory 1402) of the catheter is again stored in memory by the system controller 100 when the second fluoroscopic image 1422 is captured. The second image shows that the targeting is getting worse, so the user may take a further targeting step.

Subsequently, a third fluoroscopic image 1423 is captured at time stamp 6:54 when the operator pushes a button of the handheld controller 105 in a further attempt to realign the catheter tip with the target 1401. Here, the corresponding input command defining the posture (for trajectory 1402) of the catheter at the current positon is again stored in memory by the system controller 100 when the third fluoroscopic image 1423 is captured.

The captured images 1400 are displayed in a monitor (e.g., the main display 101-1 of the system console) in chronological order, as shown by their time stamps. By observing the series of captured images 1400, the operator can observe and easily determine the image with the best targeting accuracy. When an operator selects the one of recorded images with the best targeting accuracy, the stored corresponding input command is sent to the actuator unit 103 to recreate the posture of the steerable catheter 104 at the time when the image was captured. According to this embodiment, since the system stores the commands used for each postured, an operator can come back to a past posture of the steerable catheter by simply selecting a fluoroscopic image captured in the past targeting process. It should be appreciated by those skilled in the art, that a “past targeting process” refers to catheter controlling steps and image recording steps used to estimate, judge, explore, approximate, test or quantify various catheter postures for aligning the catheter tip with a desired target. In the various embodiments described above, the robotic catheter system 1000 may include a flexible bronchial instrument, such as a bronchoscope or bronchial catheter, for use in examination, diagnosis, biopsy, or treatment of a lung. The robotic catheter system 1000 is also suited for navigation and treatment of other tissues, via natural or surgically created bodily lumens, in any of a variety of anatomic systems, including the colon, the intestines, the urinary tract, the kidneys, the brain, the heart, the vascular system including blood vessels, and the like.

### Software Implementations

At least certain aspects of the exemplary embodiments described herein can be realized by a computer of a system or apparatus that reads out and executes computer executable instructions (e.g., one or more programs or executable code) recorded on a storage medium (which may also be referred to as a ‘non-transitory computer-readable storage medium’) to perform functions of one or more block diagrams, systems, or flowchart described above. FIG. 6 and FIG. 7A and FIG. 7B illustrate flowcharts for exemplary processes (planning 600, navigation 700, targeting S702A/S705A) of a method of operating a robotic catheter system 1000 which is configured to manipulate a steerable catheter 104 having one or more bending segments and a catheter tip 120, and which includes an actuator unit 103 coupled to the bending segments via one or more drive wires 210 arranged along a wall of the steerable catheter 104. In addition, FIG. 8 through FIG. 14 show exemplary displays, in real-time or virtual view, of estimated sampling locations based on the position and orientation of the catheter tip.

The computer may include various components known to a person having ordinary skill in the art. For example, the computer may include signal processor implemented by one or more circuits (e.g., a field programmable gate array (FPGA) or an application specific integrated circuit (ASIC)) for performing the functions of one or more of the above-described embodiment(s), by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)), and may include a network of separate computers or separate processors to read out and execute the computer executable instructions. The computer executable instructions may be provided to the computer, for example, from a cloud-based network or from the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. The computer may include an input/output (I/O) interface to receive and/or send communication signals (data) to input and output devices, which may include a keyboard, a display, a mouse, a touch screen, touchless interface (e.g., a gesture recognition device), a printing device, an stylus, an optical storage device, a scanner, a microphone, a camera, a network drive, a wired or wireless communication port, etc.

### Modifications, Combinations of Embodiments, and/or Other Embodiments

The various embodiments disclosed in the present disclosure provide several advantages over conventional targeting of robotic catheter systems. According to the embodiment, a robotic catheter system is configured to record a history of estimated locations in a targeting mode. An operator can refer to the history of accuracy targeting of the targeting mode to go back to a past posture of the steerable catheter to increase accuracy of target sampling. By displaying a history of estimated sampling locations, the operator can understand the best sampling location and the trend of the sampling among all targeting attempts by referring to the objective information. Then, the operator can make a judgment of whether to execute sampling at one of the historical sampling locations or at the current location or to continue targeting. This can reduce the procedure duration and increase targeting accuracy. Also, since the operator does not need to mentally remember the past sampling locations, and does not need to manually go back to past estimated sampling locations, it is possible reduce the operator's mental burden for targeting sampling locations that are difficult to reach. Advantageously, the user can select the sampling location with the highest targeting accuracy by simply choosing a marker that is hyperlinked to the position and orientation (pose) of the catheter tip that is best aligned with the desired target. To that end, the system can display the markers with a different color, shape, size, etc. For example, the controller 102 may control the GUI to display the marker with highest accuracy as crosshairs in green color, and the markers with lower accuracy as icons of different shapes or sizes in orange or red colors, where the green color can be an example of an acceptable color and the orange or red colors can be examples of non-acceptable or warning colors.

According to a first aspect, the present disclosure provides system having a robotic catheter having a catheter body and a distal tip. A tracking device operatively connected to the robotic catheter monitors (tracks) movement of the distal tip, and output a signal. A controller controls an actuator to bend one or more segments of catheter body to move the distal tip with respect to a target, and determines the position and orientation of the distal tip based on the signal output by the tracking device. A display device shows information about an alignment of the distal tip with respect to sampling locations within the target. The controller estimates the expected sampling locations based on the position and orientation of the distal tip. The controller stores the history of the expected sampling locations. The display device shows the history of expected the sampling locations.

According to a second aspect, the present disclosure provides a system of a display control apparatus comprising: a tracking device to identify the current position and posture of a robotic catheter used for sampling a target. A controller determines an expected position of sampling based on the identified position and posture of the catheter tip and the position of the target. A storage unit stores history of the expected positions in accordance with a transition of a position of the catheter tip. A display control unit displays the history of the expected positions with an image of a target of the sampling.

In the first or second aspect, the controller stores the input commands sent to the robotic catheter to recreate the posture of the corresponding history of the expected sampling locations.

In the first of second aspect, the tracking device includes an electromagnetic (EM) tracking sensor, and the controller determines the position and orientation of the distal tip by using the EM tracking sensor in the distal tip.

In the first or second aspect, the robotic catheter includes a bronchoscopic camera, and the controller determines the position and orientation of the distal tip by using a bronchoscopic camera view.

In the first or second aspect, the robotic catheter is imaged by a secondary imaging modality including a fluoroscopy modality, and the controller determines the position and orientation of the distal tip by using fluoroscopy images.

In the first or second aspect, the controller determines the position and orientation of the distal tip by using optical shape sensor in the robotic catheter body.

In the first or second aspect, the controller stores at least two points in the history including start and end point.

In the first or second aspect, the controller computes the closest point along the distal tip orientation from the center of the target as the sampling location with the highest accuracy of alignment.

In the first or second aspect, the controller controls the display device to show expected sampling location as markers, wherein the markers are shown with different colors/shapes/sizes based on the accuracy of alignment of the catheter tip with the target.

In the first or second aspect, the controller determines a positional relationship between the target and the expected positions of sampling within the target, and the display control unit determines at least one of color, size and shape of markers of the expected positions based on the determined positional relationship between the target and the expected positions of sampling.

In the first or second aspect, the controller determines a positional relationship between the target and a tip position of the robotic catheter, and the display control unit determines at least one of color, size and shape of a display of the expected position based on the determined positional relationship between the target and the tip position corresponding to the expected position.

In the first or second aspect, a storage device stores the history of the input commands sent to the robotic catheter with the history of the expected positions of sampling. In this case, the controller can recreate the posture of the robotic catheter using the history stored in the storage device.

In the first or second aspect, the display control apparatus is configured to provide a function for a user to select at least two positions in the display to indicate the start and end point of input commands to the robotic catheter. In this case, the controller sends the input commands to the robotic catheter to recreate the posture of the catheter at the start and end.

In the first or second aspect, the system further includes a ventilator configured to send a respiratory phase to the controller, and the controller stores the respiratory phase in a storage device. In this case, the controller sends input commands to the robotic catheter to recreate the posture at the same respiratory phase when an expected position of the sampling is selected.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by persons of ordinary skill in the art to which this disclosure belongs. In that regard, breadth and scope of the present disclosure is not limited by the specification or drawings, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. All embodiments can be modified and/or combined to improve and/or simplify the targeting process as applicable to specific applications. The scope of the following claims is to be accorded the broadest interpretation so as to encompass all such modifications, combinations, and equivalent structures and functions.

Any patent, pre-grant patent publication, or other disclosure, in whole or in part, that is said to be incorporated by reference herein is incorporated only to the extent that the incorporated materials do not conflict with standard definitions or terms, or with statements and descriptions set forth in the present disclosure. As such, and to the extent necessary, the disclosure as explicitly set forth herein supersedes any conflicting material incorporated by reference.

1-39. (canceled)

40. A method of controlling a steerable catheter, comprising:
providing a catheter body that has one or more bendable segments and a catheter tip, wherein the one or more bendable segments are actuatable by an actuator that applies an actuation force to one or more drive wires arranged along the catheter body;
inserting at least part of the catheter body into a bodily lumen such that the catheter tip is within a predetermined distance relative to a target;
controlling the actuator to actuate at least one of the bendable segments by applying a force to the one or more drive wires so as to consecutively align the catheter tip with a plurality of sampling locations in or around the target;
determining, based on the position and/or orientation of the catheter tip at each of the plurality of sampling locations, whether the catheter tip is aligned with the target;
displaying information about an accuracy of alignment of the catheter tip with respect to at least one of the plurality of sampling locations in or around the target; and
estimating an amount of accuracy of alignment between the catheter tip and each of the plurality of sampling locations based on the position and/or orientation of the catheter tip at each of the plurality of sampling locations.

41. The method of claim 40, further comprising:
storing a history of commands for aligning the catheter tip with each of the plurality of sampling locations; and
displaying the plurality of sampling locations.

42. The method of claim 40, further comprising:
for each of the plurality of sampling locations, inputting a command into the actuator to cause the actuator to align the catheter tip with the estimated sampling location; and
storing the input commands entered into the actuator to re-create the posture of the catheter tip corresponding to the history of the estimated sampling locations.

43. The method of claim 40, further comprising:
arranging a bronchoscopic camera at the catheter tip prior to inserting the catheter body into the bodily lumen;
recording a bronchoscopic camera view during the catheter insertion into the bodily lumen; and
determining the position and orientation of the catheter tip by using a bronchoscopic camera view.

44. The method of claim 40, further comprising:
displaying the position and/or orientation of the catheter tip with respect to the target as a plurality of markers,
wherein the plurality of markers are displayed with different colors/shapes/sizes based on the accuracy of alignment of the catheter tip toward the target, and
wherein at least one marker of the plurality of markers indicating a closest distance to the target is displayed with a different color/shape/size.

45. The method of claim 40, further comprising:
recording, in a memory, a history of input commands sent by the actuator to one or more of the bending segments of the catheter body, wherein the history of input commands are associated with a history of estimated positions of sampling; and
recreating the posture of the catheter tip based on the history of input commands are associated with the history of the estimated positions of sampling stored in the memory.

46. The method of claim 40, further comprising:
displaying a graphical user interface for a user to select at least two positions in the display to indicate a start point and an end point of input commands to align the catheter tip to the target,
wherein the actuator sends input commands to the catheter to re-create the posture at the start point and the end point.

47. A system, comprising:
a catheter having one or more bending segments along a length thereof and a catheter tip at the distal end thereof;
an actuator coupled to the bending segments via one or more drive wires arranged along the catheter;
a memory;
a controller configured to:
record one or more points along an insertion trajectory of at least part of the catheter inserted into a lumen along the insertion trajectory that spans from an insertion point to a target;
control the actuator to actuate at least one of the one or more drive wires to apply a force to the one or more bending segments so as to consecutively align the catheter tip with a plurality of sampling locations in or around the target;
determine, based a position and/or orientation of the catheter tip with respect to the target, an amount of accuracy of alignment of the catheter tip with respect to each of the plurality of sampling locations;
display, on one or more display screens, information about the accuracy of alignment of the catheter tip with respect to at least one of the plurality of sampling locations in or around the target;
store, in the memory, a history of input commands sent to the one or more bending segments in association with the history of the estimated positions of sampling; and
re-create the posture of the catheter using the stored history of input commands.

48. The system according to claim 47,
wherein the controller is further configured to:
estimate a sampling location of the target based on the position and orientation of the catheter tip.

49. The system according to claim 47,
wherein the controller is further configured to:
store a history of estimated sampling locations, and
control display of the history of estimated sampling locations.

50. The system according to claim 47,
wherein the controller is further configured to:
compute a closest point along the catheter tip orientation from a center of the target as the estimated sampling location.

51. The system according to claim 47,
wherein the controller is further configured to:
provide a function for a user to select at least two positions in the display to indicate a start point and an end point of the insertion trajectory, and
wherein the actuator sends the user selected points as input commands to the catheter.

52. A display control apparatus connected to a robotic catheter system which is configured to manipulate a catheter having one or more bending segments along a length thereof and a catheter tip at the distal end thereof, and which includes an actuator coupled to the bending segments via one or more drive wires arranged along the catheter, the display control apparatus comprising:
a device configured to identify one of position and posture of the catheter tip inserted in a lumen;
a controller configured to determine estimated positions of sampling of a target, based on the identified position and posture of the catheter tip with respect to the position of the target;
a memory configured to record a history of the estimated positions of sampling in accordance with a transition of a position of the catheter tip for each of the estimated positions; and
a display controller configured to display the history of the estimated positions of sampling with an image of the target.

53. The display control apparatus according to claim 52,
wherein the controller is further configured to determine a positional relationship between the target and the estimated positions of sampling,
wherein the estimated positions of sampling are represented by markers, and
wherein the display controller displays the markers according to at least one of color, size and shape based on the determined positional relationship between the target and the estimated positions of sampling.

54. The display control apparatus according to claim 52,
wherein the display controller is configured to at least one of:
display markers with different colors or shapes or sizes based on a distance between catheter tip and the target, and
display a marker indicating a location of highest accuracy with a different color or shape or size than markers indicating lower accuracy.

55. The display control apparatus according to claim 52,
wherein the display controller is configured to display a marker indicating a location of highest accuracy with a different color or shape or size than markers indicating lower accuracy.

56. The display control apparatus according to claim 52,
wherein the controller is further configured to identify at least one of the estimated positions based on a positional relationship between the target and the estimated positions; and
wherein the display controller emphasizes a display of the estimated position identified.

57. The display control apparatus according to claim 52,
wherein the memory stores a history of input commands sent to the actuator in association with the history of the estimated positions of sampling, and
wherein the controller is configured to re-create the posture of the catheter using the history of input commands stored in the memory.

58. The display control apparatus according to claim 52,
wherein the controller is further configured to prompt a user to select, among the displayed history of the estimated positions of sampling, a start point and an end point as input commands, and
wherein the controller sends the input commands to re-create the posture of the catheter at the start point and the end point.

59. The display control apparatus according to claim 52,
wherein the controller is further configured to record, in the memory, a respiratory phase of a patient, and
wherein the controller sends input commands to the actuator to re-create the posture of the catheter at the same respiratory phase when an estimated position of the sampling is selected.

60. The display control apparatus according to claim 52,
wherein the controller is further configured to:
receive one of endoscopic images from an endoscope camera arranged in the catheter tip and fluoroscopy images from a fluoroscopy modality that tracks the catheter tip, and
store, in the memory, a history of input commands with a history of the one of endoscopic images and fluoroscopy images,
wherein the display controller further displays the history of the one of endoscopic images and fluoroscopy images.

61. The display control apparatus according to claim 60,
wherein the controller is further configured to:
re-create the posture of the catheter using the history of the one of fluoroscopy images and endoscopic images stored in the memory, and
wherein the display controller displays the re-created posture of the catheter.

62. The display control apparatus according to claim 52,
wherein the memory stores a history of input commands sent by the actuator to the catheter in association with the history of the estimated positions of sampling,
wherein the memory further stores input commands input by a user to modify or replace at least one of the input commands sent by the actuator, and
wherein the controller is configured to re-create the posture of the catheter using the history of input commands modified or replaced by the input commands input by the user.

63. A system for providing visualization of a targeting trajectory for a steerable catheter relative to a target, the system comprising:
a catheter body having one or more bendable segments and a catheter tip;
an actuator configured to apply a force to the one or more bendable segments so as to consecutively align the catheter tip to a plurality of locations within a target;
a tracking system operably coupled to the catheter body and configured to collect positional information indicative of a targeting trajectory from the catheter tip to each of the plurality of locations within the target; and
a display device configured to display a graphical user interface (GUI) showing a level of accuracy of alignment of the catheter tip with each of the plurality of locations within the target,
wherein the GUI displays the level of accuracy of alignment of the catheter tip with each of the plurality of locations within the target as a marker superposed on an image of the target.
